(19) United States
(12) Patent Application Publication
(10) Pub. No.: US 2022/0074092 A1
(43) Pub. Date: Mar. 10, 2022

(54) LOW GSM FIBER WEB AND METHOD OF MAKING SAME

(71) Applicant: Preco, Inc., Somerset, WI (US)

(72) Inventor: Ken Arvey, Emerald, WI (US)

(21) Appl. No.: 17/467,935

(22) Filed: Sep. 7, 2021

Related U.S. Application Data
(60) Provisional application No. 63/075,593, filed on Sep. 8, 2020.

(51) Int. Cl. D04H 1/4266; C08B 37/00; C08L 5/04; D04H 1/46

(52) U.S. Cl. CPC D04H 1/4266; D04H 1/46; C08L 5/04; C08B 37/0084

(57) ABSTRACT

A light-weight fabric and method for producing the fabric, the method comprising providing a selected mass of starting material comprising fibers of the starting material, the fibers being in the range of about 5 cm to 30 cm in length and orienting the fibers in the selected mass to produce a loose web of fibers. The fibers in the loose web of fibers are mechanically attached or felted to provide structural integrity to the web and thus tighten the web of fibers into the fabric. The fabric is light weight and thus less than approximately 100 GSM in weight, more specifically less than approximately 50 GSM and may even be about approximately 25 GSM. The fabric can be used in wound care dressings.

### CROSS-REFERENCE TO RELATED APPLICATION

This application claims priority to U.S. Provisional Patent Application No. 63/075,593, filed Sep. 8, 2020, the disclosure of which is incorporated herein by reference in its entirety.

### BACKGROUND

The present disclosure relates to a method of producing a light-weight fabric and more specifically to a method of producing a low GSM alginate fiber fabric.

Alginate fabrics for providing surgical and medical dressings and the like have been produced from continuous calcium alginate filament yarn by needle perforations or knitting. These fabrics are provided for dressings in heavy weights including 100 grams per square meter (GSM), 150 GSM, and 300 GSM for example and above. Calcium alginate (“CA”) is a common wound care material that is derived from sea weed (Alginate powder). Conventional methods of processing the calcium alginate are limited in the production of materials having a high weight in terms of grams per square meter (GSM) due to the wide web nature of the mass production process. Many of the fabrics referred to as 100 GSM fabrics are even heavier upon analysis and 100 GSM fabrics may include fabrics up to 130 GSM.

Alginate fabrics produced in a wide web format cannot be less than 100 GSM as the machine used to produce the web controls the thickness of the web, preventing the manufacture of lower weight and/or thinner alginate fabrics. The machine technology is not capable of supporting the manufacture of light weight fabric webs.

Calcium Alginate (CA) is a raw material for a fabric that is produced in a finished mat in weights greater that 100 GSM as current methods of manufacture and machines are incapable of producing a CA fabric with structural integrity at lighter weights. CA may also be provided in the form of a TOW or rope of long continuous strands of CA. These fibers can then be needle felted or knit into a high GSM fabric as mentioned above.

### SUMMARY

An aspect of the present disclosure relates to a method for producing a light weight fabric having a weight less than approximately 100 GSM. The method comprises providing a mass of fibers having a length less than about 30 cm to a carding process to orient and arrange the fibers into a loose, non-woven web. The loose web is felted to attach and secure the fibers into a fabric. This fabric will have the body to support itself for further processing.

The mass of fibers is less than approximately 100 GSM when provided to the carding process and the mass of fibers is selected based on a selected mass of the fabric produced. The mass provided to the carding process is substantially equal to the selected mass of the fabric produced.

A starting material is provided that is calcium alginate. The calcium alginate is prepared into a mass of fibers having of a length less than about 30 cm. Raw calcium alginate may be provided in the form of a rope of long continuous strands that are cut, chopped, or otherwise reduced to a length less than about 30 cm.

The loose web may be provided with a carrier layer when transferring the loose web for felting the web to produce the fabric.

After felting, the fabric is provided in a narrow web, for example, up to 20 inches wide, which is then subsequently processed to produce one or more articles. The articles include wound care devices.

Another aspect of the present disclosure relates to a wound care device comprised of a calcium alginate fabric of less than approximately 75 GSM. The device comprises only calcium alginate fibers and the fibers are less than approximately 30 cm in length. This device construction can encapsulate antimicrobial materials between two less than 35 GSM CA webs.

The fabric is formed by a combination of a carding process and a felting process.

Another aspect of the present disclosure relates to a method for producing a fabric. The fabric is produced from a selected mass of starting material comprising fibers of the starting material, the fibers being in the range of about 5 cm to about 30 cm in length and orienting the fibers in the selected mass to produce a loose web of fibers. Mechanically attaching the fibers to one another provides structural integrity to the web and thus tightens the web of fibers into the fabric. The fabric is a light-weight fabric and is less than approximately 100 GSM in weight.

The starting material is provided in continuous strands of fibers and the fibers are cut or chopped to lengths in the range of about 5 cm to about 30 cm.

Orienting the fibers comprises providing the selected mass of starting material to a carding machine to produce the loose web of fibers.

Mechanically attaching the fibers comprises felting the loose web of fibers.

The selected mass has a weight that is substantially equal to the GSM weight of the produced fabric. In one or more embodiments, the GSM weight of the fabric is less than approximately 75 GSM. In one or more embodiments, the GSM weight of the fabric is less than approximately 30 GSM.

A carrier can be provided for supporting the loose web of fibers prior to or during mechanical attachment of the fibers to form the fabric.

The starting material is calcium alginate fibers. The calcium alginate fibers may be produced from sodium alginate or other power materials.

Another aspect of the present disclosure relates to a calcium alginate fabric with a weight less than approximately 100 GSM comprises of calcium alginate fibers having a length in the range of about 5 cm to about 30 cm and wherein the calcium alginate fibers are felted such that the fabric is a non-woven fabric.

The fabric has a weight less than approximately 30 GSM.

Yet another aspect of the present disclosure relates to an article constructed from a calcium alginate fabric with a weight less than approximately 75 GSM comprises of calcium alginate fibers having a length in the range of about 5 cm to about 30 cm and wherein the calcium alginate fibers are felted such that the fabric is a non-woven fabric wherein the article is a wound care dressing.

In one or more embodiments, the fabric is a non-woven woven fabric.

### BRIEF DESCRIPTION OF THE DRAWINGS

FIG. 1 illustrates a mass of calcium alginate fibers being fed to a carding machine.

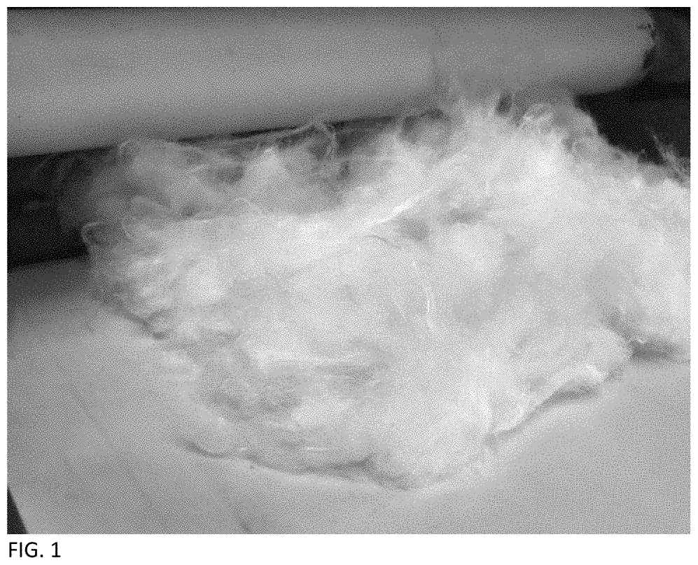

FIG. 2 illustrates a loose web produced by the carding machine comprising the mass of fibers fed to the carding machine shown in FIG. 1.

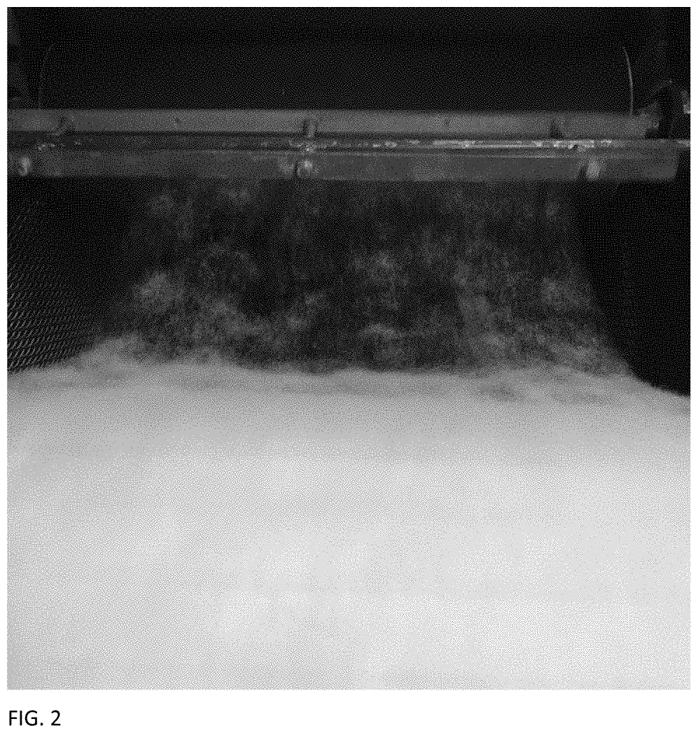

### DETAILED DESCRIPTION

Methods described herein produce a light-weight alginate fabric. In one embodiment, a light-weight fabric of calcium alginate is produced wherein the calcium alginate fabric is less than approximately 100 grams per square meter (GSM) in weight. The calcium alginate fabric produced by the methods described herein may be less than about approximately 50 GSM, less than about approximately 30 GSM, or less than about approximately 25 GSM. Thus, the fabric produced by the methods described herein has a thickness less than the prior art methods of producing fabrics or other materials from alginate, including calcium alginate.

The methods described herein may utilize a process that uses an open or mesh conveyor concept or may use a screen to deposit prepared fibers onto the conveyor mesh at a selected or target GSM, for example, a GSM in the range of about 5 GSM to about 50 GSM or the range of about 10 to about 25 GSM. Once deposited at the target GSM, the fibers in a loose web can be needle felted, or the fibers otherwise mechanically attached to maintain stability and form the fabric. It is also contemplated and within the scope of this disclosure that the loose web of material be transferred onto a carrier liner without attachment of fibers, and rolled into a rolled good that can be robustly handled and processed.

To produce the low GSM fabric, a starting material or input material may first require cutting, chopping, or brushing to prepare fibers for the fabric, carding, or other established fiber handling methods, or combinations thereof prior to felting the material to produce the low GSM alginate fabric.

As used throughout this disclosure, the term “fiber” refers to material having a shape wherein one dimension is significantly larger than the other two dimensions and where the material is sufficiently flexible so as to enable entanglement with other fibers creating a three-dimensional network with sufficient strength to be provided as a web of material for use in a given application.

Examples of material fibers suitable for the methods and articles described herein include, but are not limited to bio-neutral fibers such as polyolefin medical grade fiber, bio-neutral and medically functional fibers such as absorbent medical grade cotton or silk, bio-absorbable fibers such as or polycaprolactones and polylactides, and bioactive and bio-absorbable fibers, such as alginate including calcium alginate, collagen, hyaluronic acid, silk fibroin, crosslinked chitosan, as well as combinations thereof and/or like fibers.

As used throughout this disclosure, the term “felting” refers to the process of producing a felted article. A mechanics operation is used to interlock fibers together to produce a non-woven article or “fabric” of sorts where the fabric is composed of interlocked fibers. While a method of needle punching for felting of the fibers to form a cohesive, non-woven article is described herein, other methods of felting or producing a mat of fibers may be used. What is meant by the term “felted” as used throughout this disclosure is akin to felting or making into felt, or matting together such that the fibers described herein are caused to adhered and mat together to form a sort of cloth comprised of the fibers as described herein. To accomplish felting, a felting needle(s) is used. Felting needles as defined herein may have a split pointed (forked) end for catching fibers to push the fibers through other fibers thereby entangling the fibers. The felting needle(s) may also have notches or barbs, positioned along the needle's shaft to catch fibers as the needle is pushed through but such barbs or notches configured so as not to catch the fibers as the needle is pulled back through the fibers. Of course felting needles may have both a split pointed end and barbs or notches. A needle felting machine may also be used for felting materials in large dimensions.

While needle felting or needle punching is a known fiber binding technology, the methods described herein with the materials described herein results in a light-weight fabric of lower GSW than the prior art methods are capable of producing with the materials described herein. This provides an additional material for use in wound care that is improved, safe, produced via a cost-effective method that produces a much thinner and lighter fabric material that in one or more embodiments eliminates the need for additional materials such as adhesives or solvents, which are not desirable components in materials for most medical applications.

In one embodiment, a selected weight of short lengths of calcium alginate fibers are disentangled, oriented, cleaned, and/or intermixed to produce a continuous web or sliver suitable for subsequent processing, otherwise referred to as a carding process. Carding is a mechanical process achieved by passing the fibers between differentially moving surfaces covered with card clothing. A carding machine may be used. The starting material may need to be prepared for carding first by cutting the fibers into shorter length before carding the selected weight of fibers. For example, if using calcium alginate TOW or rope, the strands are first cut or chopped to shorter lengths before carding.

Once carded, the material is then subject to a felting process to finish the fabric thus providing structural integrity to the fabric and strength. The fabric can then be rolled in large sheets for subsequent cutting into selected size and shape articles. To effectively supply the light weight material to the felting process in one or more embodiments, the fibers are be pre-conditioned to a selected length prior to carding and redeposited onto a roll after carding or directly into a felting machine or felting apparatus to provide stability to the web thus forming a fabric. The felting process needle bonds the fibers to produce the light-weight fabric.

While this disclosure refers specifically to calcium alginate fibers forming a fabric, it should be understood that the methods described herein can be applied to produce a low GSM or light-weight fabric from various natural and synthetic fibers, not limited to alginates.

### ILLUSTRATIVE EXAMPLE

A thick loosely wound continuous rope of calcium alginate (e.g., TOW) is provided as a starting or input material. The TOW is prepared by cutting or chopping and is prepared into a mass of fibers of selected lengths ranging from about 5 cm to about 30 cm for processing. In some embodiments, longer fibers such as those closer about 20 cm to about 30 cm or longer may appear to more efficiently and/or consistently produce a low GSM fabric material capable of being wound onto a roll and/or cut to selected sizes while retaining its fabric construction. However, smaller fibers of about 5 cm or larger can produce low GSM fabric materials of structural integrity.

To align and orient the prepared fibers, the prepared fibers are fed into a carding machine. The weight of prepared fibers is selected and may be approximately equal to a target output weight desired for the final fabric/material GSM. The feeding of the prepared fibers into the carding machine may be controlled such that the fibers are output into or in the form of a web of selected dimensions, such as selected web-width which may be set or limited based on the width of the carding machine.

Carding machines have the ability to return the fibers back into a TOW or rope format or to wind the material into a very loose fiber web. Thus, settings are selected which produce a loose fiber web. The web can then be supported using a carrier liner as the fibers are simply oriented in the manner of the fabric, but are not attached or connected so as to make a fabric.

To form the fibers into a web or fabric that can be handled more robustly, the carded web is fed into a felter such as a needle felting machine. The needle felting machine forms the fabric from the carded fabric web by transforming the loose web into a tangible fabric like material of a GSW less than 100. The fibers become bonded through the felting process. More specifically, this method successfully delivered prepared fibers to the carding and felting machines such that approximately 31.29 GSM CA fabric is output in a fashion that is robust enough to be post processed into articles such as one or more types of wound care devices, such as dressings. The fabric may be manufactured into articles competitive with Combat Gauze encapsulating Bio-Glass scaffold materials into two layers of CA yielding a total structure below approximately 75 GSM. The fabric can be formed having about a 35 GSM weight and this fabric can also cut and/or formed into a wound care dressing or otherwise used for wound care at said weight.

In some embodiments, a water mist or Isopropyl Alcohol can be used to “tack” the fibers together to assist in the transfer of the loose web from the carding process to the felting process. However, in one or more embodiments described herein no additional materials are introduced to the mass of raw fibers such as the CA fibers for one or more physical, biological and processing reasons based on end use of the fabric for example.

Additional or alternative methods of preparing and/or dispensing the fiber mass for carding for example can be used, including but not limited to fiberglass choppers or sprayers. Without being bound by theory, it is believed that the methods of preparing and providing the fibers to the carding process as described herein result in an increased ability to tightly control the fiber size and mass consistency. For example, calcium alginate is more ductile than the brittle fibers of fiberglass and thus react the preparing processes described herein differently and can be efficiently and accurate cut or chopped as described previously above.

Various articles may be produced from the fabric material produced according to one or more of the methods described herein. The articles may be cut from the fabric in various shapes and sizes fabrics produced according to methods described herein, and further, these layers maybe be used to encapsulate additional materials or components. While calcium alginate fabrics of less than approximately 100 GSM, less than approximately 75 GSM, less than approximately 50 GSM, less than approximately 40 GSM and less than approximately 30 GSM by way of non-exhaustive example can be formed by the methods described herein, these fabrics may be used to form wound care devices including but not limited to bandages and absorption devices.

Further, felting the fibers as described herein produces a fabric that can be used to construct article that can be provided in various selected lengths, widths, densities, thicknesses, and as single or multi-layer articles. The articles may retain and wick water, oil, blood, and other liquids. The fabric is sufficiently felted to resist wear and tearing and hold its edges such that the article will not unravel when cut but the fabric is sufficiently light-weight to increase the variety of suitable articles and applications.

The articles described herein may be used in a variety of medical applications including, but not limited to, wound bandages or packing for primary wound treatment in the field; permanent wound treatments, packing, or scaffolding that remains in place and is eventually reabsorbed by the body.

The fabric described herein and/or produced by the methods described herein may be provided as a continuous non-woven web that can be handled without disintegrating and wherein the web can be cut and/or shaped into forms useful for medical applications. The articles are felted in that the articles are non-woven materials of interlocked fibers.

Although the present disclosure has been described with reference to preferred embodiments, workers skilled in the art will recognize that changes may be made in form and detail without departing from the spirit and scope of the disclosure.

1. A method for producing a fabric, the method comprising:
providing a selected mass of starting material comprising fibers of the starting material, the fibers being in the range of about 5 cm to about 30 cm in length;
orienting the fibers in the selected mass to produce a loose web of fibers; and
mechanically attaching the fibers to provide structural integrity to the web and thus tighten the web of fibers into the fabric,
wherein the fabric is less than approximately 100 GSM in weight.

2. The method of claim 1, wherein the starting material is provided in continuous strands of fibers and the fibers are cut or chopped to lengths in the range of about 5 cm to about 30 cm.

3. The method of claim 1 wherein orienting the fibers comprises providing the selected mass of starting material to a carding machine to produce the loose web of fibers.

4. The method of claim 1 wherein mechanically attaching the fibers comprises felting the loose web of fibers.

5. The method of claim 1, wherein the selected mass has a weight that is substantially equal to the GSM weight of the produced fabric.

6. The method of claim 5, the GSM weight of the fabric is less than approximately 75 GSM.

7. The method of claim 5, the GSM weight of the fabric is less than approximately 30 GSM.

8. The method of claim 1, and further providing a carrier for supporting the loose web of fibers prior to or during mechanical attachment of the fibers to form the fabric.

9. The method of claim 1 and producing an article from the fabric wherein the article is a wound care device.

10. The method of claim 9, wherein the article is non-woven.

11. The method of claim 9 wherein the starting material is calcium alginate fibers.

12. A calcium alginate fabric with a weight less than approximately 100 GSM comprised of calcium alginate fibers having a length in the range of about 5 cm to about 30 cm and wherein the calcium alginate fibers are felted such that the fabric is a non-woven fabric.

13. The fabric of claim 12 with a weight less than approximately 30 GSM.

14. An article constructed from a calcium alginate fabric with a weight less than approximately 75 GSM comprises of calcium alginate fibers having a length in the range of about 5 cm to about 30 cm and wherein the calcium alginate fibers are felted such that the fabric is a non-woven fabric wherein the article is a wound care dressing.
